(19) United States
(12) Patent Application Publication
(10) Pub. No.: US 2019/0134047 A1
(43) Pub. Date: May 9, 2019

(54) A MIXTURE OF D-SERINE AND CAFFEINE FOR OBTAINING THE EFFECT OF PREVENTING THE DECLINE OF LEARNING ABILITY OR MEMORY CAUSED BY STRESS AND ENHANCING LEARNING ABILITY OR MEMORY, AND A FUNCTIONAL FOOD COMPOSITION CONTAINING THE SAME

(71) Applicants: ADVANCED NT, Seoul (KR); ANT LABS INC, Seoul (KR)

(72) Inventors: Kon CHU, Seoul (KR); Sang Kun LEE, Seoul (KR); Keun Hwa JUNG, Seoul (KR); Soon Tae LEE, Seoul (KR); Man Ho KIM, Seoul (KR); Dae Jong JEON, Daejeon (KR)

(73) Assignees: ADVANCED NT, Seoul (KR); ANT LABS INC, Seoul (KR)

(21) Appl. No.: 16/096,728

(22) PCT Filed: Apr. 25, 2017
(86) PCT No.: PCT/KR2017/004348

§ 371 Date: Oct. 26, 2018

(30) Foreign Application Priority Data
Apr. 27, 2016 (KR) 10-2016-0051659

(51) Int. Cl. A61K 31/522; A61K 31/198; A61K 9/00; A61P 25/22; A23L 33/175; A23L 33/155; A23L 33/105

(52) U.S. Cl. CPC A61K 31/522; A23V 2250/712; A61K 9/0053; A61P 25/22; A23L 33/175; A23L 33/155; A23L 33/105; A23V 2002/00; A23V 2200/322; A23V 2250/0642; A23V 2250/2108; A23V 2250/702; A23V 2250/704; A23V 2250/708; A23V 2250/71; A61K 31/198

(57) ABSTRACT

Provided is a mixture containing D-serine and caffeine as an active ingredient for obtaining the effect of preventing the decline of learning ability or memory caused by stress, reducing the influence of stress and enhancing learning ability or memory, and to provide a functional food composition containing the same.

### BACKGROUND OF THE INVENTION

### Field of the Invention

The present invention relates to a mixture containing D-serine and caffeine as active ingredient for obtaining the effect of preventing the decline of learning ability or memory caused by stress and enhancing learning ability or memory, and a functional food composition containing the same.

### Description of the Prior Art

In general, at a highly developed society, modern people are known to be subjected with a stress by adults as well as infants and young people. Excessive stress can also cause brain diseases such as br Ong and depression. In addition, this may result in decreased learning ability or memory capacity.

Especially, the decline of learning ability and memory has caused problems for modern people to stress more and more heavily. In addition, excessive stress in childhood can lead to a decline in learning and memory when grown up as an adult. Therefore, there is a need to suppress the decline of the learning ability or memory ability due to the stress.

On the other hand, in relation to brain function, L-serine is generally converted to D-serine by serine racemizing enzyme in astrocytes, acting as a neurotransmitter in the synapse. Also, it is known that the concentration of D-serine in the brain increases through the blood-brain-bather (BBB) when supplied externally. Such D-serine are thought to influence learning ability and memory by stimulating synaptic transmission and neuronal plasticity by stimulating NMDA receptors present in synapses.

And, caffeine is a major function of inhibiting adenosine binding to adenosine receptors and influences the inositol triphosphate (IP3) receptor or ryanodine receptors (RYRs) so that it also has the ability to control the concentration of intracellular calcium. When caffeine is absorbed into the body, it stimulates the adrenal gland to secrete the hormones adrenaline and noradrenaline, thereby increasing the activity of the brain, heart, skeletal muscle, and kidney, and improving the alertness, fatigue and mental clarity.

However, caffeine acts on the central nervous system to cause symptoms such as anxiety, irritancy, nervousness, excitement, insomnia, or breathing difficulties associated with muscle movement and cause heart disease.

As described above, brain-related effects of D-serine and caffeine are known respectively. However, detailed effects on the prevention of decline of learning ability or memory caused by stress and improvement of learning ability or memory are not known, and in particular, there is a lack of research on effects on the human body in the case of mixed use.

### SUMMARY OF THE INVENTION

Accordingly, it is an object of the present invention to solve the above problems, and to provide a functional food composition which is prepared from a mixture containing D-serine and caffeine as an active ingredient produced from a process for mixing D-serine and caffeine in an appropriate ratio and has an effect of inhibiting decline of learning ability or memory caused by stress and an effect of improving learning ability or memory.

In order to achieve the above object, the mixture according to the embodiment of the present invention includes a composition containing mixed D-serine and caffeine as an active ingredient, having an effect of preventing the decline of learning ability and memory caused by stress.

Also, the said composition according to the present invention is characterized by containing D-serine and caffeine at a constant weight ratio.

Also, the said composition according to the present invention is characterized by containing 50 mg of D-serine and 10 mg of the caffeine.

Also, the said composition according to the present invention is characterized by containing 500 mg of D-serine and 10 mg of the caffeine.

In addition, the said composition according to the present invention is characterized by containing D-serine and caffeine in a weight ratio of 5:1.

Further, the mixture according to the present invention is characterized in that it comprises at least one vitamin selected from the group consisting of vitamin A complex, vitamin B complex, vitamin C, vitamin D and vitamin E.

The mixture according to an embodiment of the present invention includes a mixture containing mixed D-serine and caffeine composition as an active ingredient, reducing the influence of stress, and enhancing learning ability and memory.

Also, the said mixture according to the present invention is characterized in that it comprises at least one vitamin selected from the group consisting of vitamin A complex, vitamin B complex, vitamin C, vitamin D and vitamin E.

In addition, the functional food according to the embodiment of the present invention is manufactured from a mixture containing the mixed composition containing D-serine and caffeine as an active ingredient, and has a function of preventing the decline of learning ability and memory caused by stress.

In addition, the said functional food according to the present invention is characterized in that it is selected from the group consisting of drinks, pills, granules, tablets, syrups and capsules.

The functional food according to the embodiment of the present invention may be manufactured from a mixture comprising the mixed composition containing D-serine and caffeine as an active ingredient, and has a function of reducing the influence of stress and improving learning ability and memory.

Being constituted as the above, according to an embodiment of the present invention, a mixture containing D-serine and caffeine as an active ingredient can be provided to prevent or suppress the decline of learning ability or memory caused by stress.

Further, it is possible to provide a mixture containing D-serine and caffeine as an active ingredient according to the embodiment of the present invention, thereby reducing the influence of stress and improving learning ability or memory.

On the other hand, according to an embodiment of the present invention, a functional food composition containing a mixture comprising D-serine and caffeine as an active ingredient is prepared, thereby reducing the influence of stress and improving learning ability or memory.

### BRIEF DESCRIPTION OF THE DRAWINGS

FIG. 1 is a histogram of the effect of D-serine, caffeine, and D-serine and caffeine mixtures on restraint stress in behavioral experiments measuring Novel object recognition memory.

FIG. 2 is a bar graph showing the results of an elevated plus maze behavior test measuring anxiety after injecting a mixture of D-serine and caffeine.

### DETAILED DESCRIPTION OF THE INVENTION

Hereinafter, the present invention will be described in further detail with reference to preferred embodiments. It is to be understood, however, that the scope of the present invention is not limited to these embodiments. It should be noted that the embodiments of the present invention described below are intended to sufficiently convey the spirit of the present invention to those skilled in the art.

As used herein, the term “mixture” may further contain one or more active ingredients that exhibit the same or similar function with respect to learning ability or memory or a compound that maintains/increases the solubility and/or absorbency of the active ingredient, other than a mixture capable of inhibiting the decline of the learning ability or memory or improving the learning ability or memory according to the present invention.

And, the functional food composition containing the said mixture may be a health supplement foods that can prevent the decline of the learning ability or memory caused by stress, or enhance the learning ability or memory that can be produced by any one of beverage, pills, granule, tablet, syrup or capsule.

According to one embodiment of the present invention, a mixture containing D-serine and caffeine in combination as an active ingredient can be prepared, which has the effect of preventing the decline of learning ability and memory caused by stress.

Also, the said composition according to the present invention is characterized by containing D-serine and caffeine at a constant weight ratio.

Also, the said composition according to the present invention is characterized by containing 50 mg of D-serine and 10 mg of the caffeine.

Also, the said composition according to the present invention is characterized by containing 500 mg of D-serine and 10 mg of the caffeine.

In addition, the said composition according to the present invention is characterized by containing D-serine and caffeine in a weight ratio of 5:1.

Further, the said mixture is characterized in that it comprises at least one vitamin selected from the group consisting of vitamin A complex, vitamin B complex, vitamin C, vitamin D and vitamin E.

According to an embodiment of the present invention, it includes a mixture containing mixed D-serine and caffeine composition as an active ingredient, reducing the influence of stress, and enhancing learning ability and memory.

Also, the said mixture is characterized in that it comprises at least one vitamin selected from the group consisting of vitamin A complex, vitamin B complex, vitamin C, vitamin D and vitamin E.

In addition, the functional food according to one embodiment of the present invention is manufactured from a mixture containing the mixed composition containing D-serine and caffeine as an active ingredient, and has a function of preventing the decline of learning ability and memory caused by stress.

In addition, the said functional food is characterized in that it is selected from the group consisting of drinks, pills, granules, tablets, syrups and capsules.

The functional food according to one embodiment of the present invention may be manufactured from a mixture comprising the mixed composition containing D-serine and caffeine as an active ingredient, and has a function of reducing the influence of stress and improving learning ability and memory.

The experimental steps used for embodiments of the present invention can be described as follows.

1. Animals and Experimental Groups

In all experiments, C57BL/6 male mouse, a kind of B6 mouse of 8-12 weeks old, were used. The experimental groups were divided into 7 experimental groups according to whether they were stressed or not and the type and weight of injected drugs as follows.

1) Normal group: being injected with saline and not applied stress.

2) Stress Control: being injected with saline, and applied stress.

3) Caffeine group: being injected with caffeine 10 mg, and applied stress.

4) D-serine (first weight) group: being injected with 50 mg of D-serine, and applied stress.

5) D-serine (second weight) group: being injected with 500 mg of D-serine, and applied stress.

6) D-serine (first weight) group/caffeine group: being injected with 50 mg of D-serine and 10 mg of caffeine together, and applied stress.

7) D-serine (second weight) group/caffeine group: being injected with 500 mg of D-serine and 10 mg of caffeine together, and applied stress.

2. Drug Injection and Stress Method

For the experiment, a mixture of saline, D-serine, caffeine and a mixture of D-serine and caffeine (first and second weights) was injected into the mouse by abdominal injection at 30 minutes before each of the behavioral experiments. Immediately after each behavioral experiment, restraint stress was applied by placing the mouse in a 50 mL tube pre-pierced with about 9 pores for 90 minutes.

3. Behavioral Experiment

The following two behavioral tests were performed to confirm the influence of the drugs used on each experimental group on learning ability or memory.

1) Novel Object Recognition Test for New Objects

A mouse was placed in an open-field box (40 cm×40 cm×40 cm) made of acryl for 15 minutes every day for 3 days to fit and stabilize to the box. On the training day of the fourth day, two different objects made of acryl (objects A and B) were placed at regular intervals in the box and the mice were trained to freely search objects A and B for 10 minutes. On the fifth day (24 hours later), one of the objects A and B was replaced with a new object (object C), and the mice were again searched for 5 minutes. The search time for each object (A and C or B and C) was quantified and the preference for the object was expressed as a ratio of search time (Preference (%)=C/(A+C)*100). The drug was injected into the mice at 30 minutes before the start of the experiment on the fourth day.

2) Elevated Plus Maze (EPM) Test

The EPM (Elevated plus maze) is made of acryl, and a cross (+) maze is made 40 cm from the bottom. Two opposing pathways of the four pathways of the maze are opened (open arms) and two opposing pathways (closed arms) are surrounded by a 20 cm high wall. The center platform was 8 cm in height length and 8 cm in wide length. At the start of the experiment, the mouse was placed on the center platform and the maze was freely searched for 5 minutes. Then, the time spent in the open and closed arms of the mouse was analyzed, and the EPM (Elevated plus maze) test was performed by injecting the drug only without applying stress to the mouse.

4. Data Analysis and Statistics

To compare the results of the said two behavioral experiments, SPSS 21.0 (SPSS Inc, Chicago, Ill.) and Student's T-test and one-way ANOVA were used for statistical analysis. All results were expressed as mean±standard error, and P-values were significantly considered to be less than 0.05.

### <Example 1> Ascertainment of Increase and Suppression of Decline in Stress-Induced Object Cognitive Ability by a Mixture Containing D-Serine and Caffeine as Active Ingredients

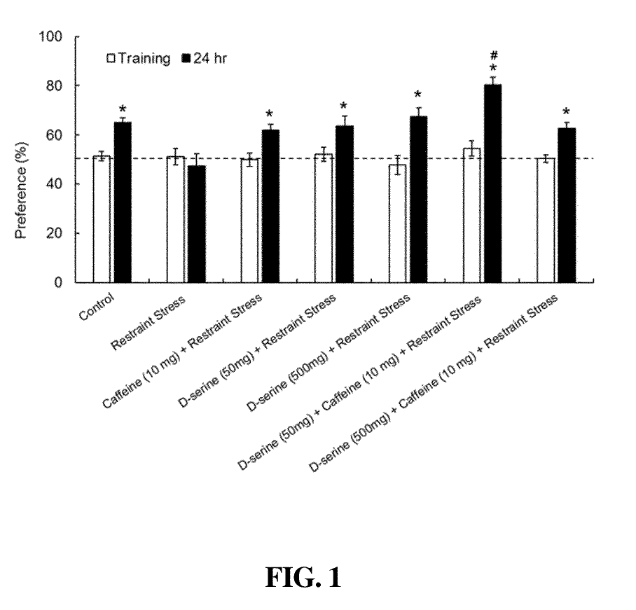

Experiments related to the cognitive behavior of new objects were performed with the above seven experimental groups. FIG. 1 is a bar graph showing preferences for new objects in seven experimental groups. The abscissa of the bar graph shown in FIG. 1 represents a normal group (Control, 10 mouse), a stress control group (Restraint stress, 9 mouse), a caffeine group (Caffeine 10 mg+Restraint Stress, 8 mouse), D-Serine (first weight) group (D-Serine 50 mg+Restraint Stress, 10 mouse), D-Serine (second weight) group (D-Serine 500 mg+Restraint Stress, 10 mouse), D-Serine (first weight)/caffeine group (D-Serine 50 mg+Caffeine 10 mg+Restraint Stress, 9 mouse), D-Serine (second weight)/caffeine group (D-Serine 500 mg+Caffeine 10 mg+Restraint Stress, 11 mouse) from left, respectively. And, the vertical axis of the bar graph shown in FIG. 1 represents the preference (%).

In the graph of FIG. 1, the white bar indicates the preference for the objects A and B of the mouse according to the seven experiment groups on the fourth day (training day), and the black bar indicates the preference for the newly disposed objects C on the fifth day (24 hours after the training).

The control group showed preference for 51.44±1.94% of objects A and B on the fourth day, and 64.96±2.04% of new object C on the fifth day after 24 hours. It can be seen from the above results that, in the case of the normal group in which the stress is not applied, when the new object C is disposed, the recognition and search time for the new object C are increased and the preference is also increased.

On the other hand, the restraint stress group showed preference for 51.20±3.26% of objects A and B on the fourth day, but 47.30±5.05% of new object C on the fifth day after 24 hours. As a result, it was confirmed that, in the restraint stress control group, when a new object C is disposed, the recognition and search time for the new object C are decreased and the preference is also decreased.

With a comparison of the results of the normal control and the restraint stress group shown in FIG. 1, it could be confirmed that when the subject receives stress, recognition and search time for a new objects are reduced, thereby causing problems in learning and memory.

The caffeine group (Caffeine 10 mg+Restraint stress) showed preference for 49.92±2.75% of objects A and B on the fourth day, but the preference for 62.06±2.27% of new object C on the fifth day after 24 hours.

The D-serine (first weight) group (D-serine 50 mg+Restraint stress) showed preference for 52.12±2.81% of objects A and B on the fourth day, but the preference for 63.63±3.94% of new object C on the fifth day after 24 hours.

The D-serine (second weight) group (D-serine 500 mg+Restraint stress) showed preference for 47.83±3.97% of objects A and B on the fourth day, but the preference for 67.55±3.46% of new object C on the fifth day after 24 hours.

The D-serine (first weight)/caffeine group (D-serine 50 mg+Caffeine 10 mg+Restraint stress) showed 54.57±3.08% preference for objects A and B on the fourth day, but the preference for 80.25±3.11% of new object C on the fifth day after 24 hours.

The D-serine (second weight)/caffeine group (D-serine 500 mg+Caffeine 10 mg+Restraint stress) showed 50.40±1.54% preference for objects A and B on the fourth day, but the preference for 62.65±2.46% of new object C on the fifth day after 24 hours.

With a comparison of the bar graph of caffeine group (Caffeine 10 mg+Restraint Stress), D-Serine (first weight) group (D-Serine 50 mg+Restraint Stress), D-Serine (second weight) group (D-Serine 500 mg+Restraint Stress), D-Serine (first weight)/caffeine group (D-Serine 50 mg+Caffeine 10 mg+Restraint Stress), D-Serine (second weight)/caffeine group (D-Serine 500 mg+Caffeine 10 mg+Restraint Stress) shown in FIG. 1, it could be confirmed that D-Serine (first weight) group (D-Serine 50 mg+Restraint Stress) showed the highest preference for new object C, thereby suppressing deterioration of learning ability or memory caused by stress with a mixture comprising a first weight of D-serine and caffeine as an active ingredient rather than a mixture comprising the second weight of D-serine and caffeine as active ingredients and also further increasing the learning ability or memory for object recognition (*P<0.01, comparison within groups, Student's Hest, #P<0.01, comparison among groups with 24-hour results, one-way ANOVA).

Thus, in the experimental group in which a mixture of the first weight of D-serine and caffeine was injected, it could be confirmed that the learning ability or memory decline caused by the stress was remarkably prevented or prevented, and learning ability or memory ability for object recognition was significantly improved. In particular, it was confirmed that the mixture containing the first weight of D-serine and the corresponding weight ratio of caffeine as an active ingredient was the most effective.

With a comparison of the preference of the stressed experimental group with that of the experimental group injected with a mixture of the first weight of D-serine and caffeine or the second weight of D-serine and caffeine, it could be confirmed that, when a mixture of D-serine and caffeine is injected, the preference is rather increased even though stress was applied. From this result, it was confirmed that a mixture of D-serine and caffeine having different weight ratios has also an effect of reducing the influence of stress on learning ability or memory.

Therefore, from the said experiment, it could be confirmed that the method of injecting the mixture of D-serine and caffeine is effective in preventing the decline of the learning ability or memory caused by stress and improving the learning ability or memory even if the weight ratio of D-serine and caffeine is different, so that it is more preferable than the method of administering D-serine and caffeine respectively. In addition, it was confirmed that the case containing D-serine and caffeine in a weight ratio of 5:1 was the most effective.

In addition, when a functional food composition containing a mixture of D-serine and caffeine according to an embodiment of the present invention is prepared, it is most preferable that a functional food having an effect to prevent decline of the learning ability or the memory caused by the stress or to improve the learning ability or memory is prepared from a mixture having a weight ratio of D-serine and caffeine of 5:1.

In addition, the said functional food may be one or more functional foods for oral use selected from the group consisting of drinks, pills, granules, tablets, syrups and capsules.

In addition, the above-mentioned mixture or functional food may contain, in addition to a composition containing D-serine and caffeine, various nutrients, vitamins, minerals (electrolytes), a flavoring agent such as synthetic flavors and natural flavors, coloring agents, carbonating agent used in carbonated beverages, and the like.

At this time, the vitamins may include one or more vitamins selected from the group consisting of vitamin A complex, vitamin B complex, vitamin C, vitamin D and vitamin E.

### <Example 2> Influence of Anxiety by D-Serine/Caffeine Mixture

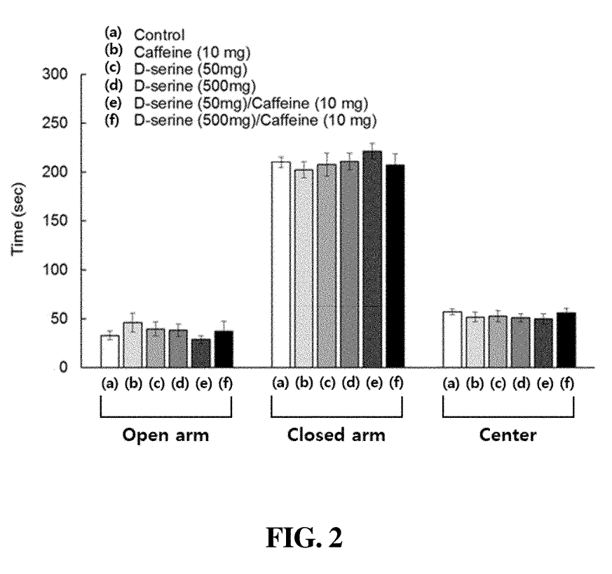

The degree of depression of the five experimental groups was measured through an EPM (Elevated plus maze) experiment performed substantially the same as the above described experimental method, thereby confirming that D-serine, caffeine, D-serine (first weight, second weight)/caffeine mixture had an effect on the formation of anxiety. The results are shown in the bar graph shown in FIG. 2.

The abscissa of the bar graph in FIG. 2 represents an open arm, a closed arm and a center platform, and a vertical axis represents staying time (sec) at each passage. A normal group (Control, 11 mouse), a caffeine group (Caffeine 10 mg+Restraint Stress, 7 mouse), D-Serine (first weight) group (D-Serine 50 mg+Restraint Stress, 9 mouse), D-Serine (second weight) group (D-Serine 500 mg+Restraint Stress, 10 mouse), D-Serine (first weight)/caffeine group (D-Serine 50 mg+Caffeine 10 mg+Restraint Stress, 7 mouse), D-Serine (second weight)/caffeine group (D-Serine 500 mg+Caffeine 10 mg+Restraint Stress, 7 mouse) were shown in order from the left side of the open arm portion of FIG. 2. And, the closed arm and the central platform are also the same as the bar sequence described in the said open arm.

From the bar graph of FIG. 2, it could be conformed that the mice of all experimental groups spent similar time at the open arm (F=0.61, P=0.69), the closed arm (F=0.41, P=0.84) and the central platform (F=0.36, P=0.87) (comparison among groups, one-way ANOVA).

With regard to whether or not adverse effects can be formed on the human body when D-serine and caffeine are mixed and used, the present experiment shows that even if the weight ratio of D-serine and caffeine injected is different before the experiment, the anxiety index of the mice is not significantly affected. Therefore, it can be seen that even when D-serine and caffeine are mixed then injected, they do not have negative effects such as formation of anxiety and the like.

Although the present invention has been particularly shown and described with reference to exemplary embodiments thereof for illustrative purposes, it is clearly understood that the same is by way of illustration and example only and is not to be construed to the preferred embodiments of the present invention, and that various modifications, additions and substitutions are possible, without departing from the scope and spirit of the invention as disclosed in the accompanied claims.

1. A mixture having an effect of preventing the decline of learning ability and memory caused by stress, comprising a composition containing mixed D-serine and caffeine as an active ingredient.

2. The mixture having an effect of preventing the decline of learning ability and memory caused by stress of claim 1, wherein the said composition contains D-serine and caffeine at a constant weight ratio.

3. The mixture having an effect of preventing the decline of learning ability and memory caused by stress of claim 1, wherein the said composition contains 50 mg of D-serine and 10 mg of the caffeine.

4. The mixture having an effect of preventing the decline of learning ability and memory caused by stress of claim 1, wherein the said composition contains 500 mg of D-serine and 10 mg of the caffeine.

5. The mixture having an effect of preventing the decline of learning ability and memory caused by stress of claim 1, wherein the said composition contains D-serine and caffeine in a weight ratio of 5:1.

6. The mixture having an effect of preventing the decline of learning ability and memory caused by stress of claim 1, wherein the said composition further comprises at least one vitamin selected from the group consisting of vitamin A complex, vitamin B complex, vitamin C, vitamin D and vitamin E.

7. A mixture having an effect of reducing the influence of stress, and enhancing learning ability and memory, comprising a composition containing mixed D-serine and caffeine as an active ingredient.

8. The mixture having an effect of enhancing learning ability and memory of claim 7, wherein the said composition further comprises at least one vitamin selected from the group consisting of vitamin A complex, vitamin B complex, vitamin C, vitamin D and vitamin E.

9. A functional food having an effect of preventing the decline of learning ability and memory caused by stress, being manufactured from a mixture containing the mixed composition containing D-serine and caffeine as an active ingredient.

10. The functional food having an effect of preventing the decline of learning ability and memory caused by stress of claim 9, the said functional food is selected from the group consisting of drinks, pills, granules, tablets, syrups and capsules.

11. (canceled)
